(19) United States
(12) Patent Application Publication
(10) Pub. No.: US 2009/0292146 A1
(43) Pub. Date: Nov. 26, 2009

(54) CATALYST PRECURSOR FOR AN Rh COMPLEX CATALYST

(75) Inventors: Dieter Hess, Marl (DE); Klaus-Diether Wiese, Haltern am See (DE); Oliver Moeller, Oer-Erkenschwick (DE); Dirk Fridag, Haltern am See (DE); Detlef Selent, Rostock (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: Evonik Oxeno GmbH, Marl (DE)

(21) Appl. No.: 12/373,921

(22) PCT Filed: May 29, 2007
(86) PCT No.: PCT/EP2007/055165

§ 371 Date: Jan. 15, 2009

(30) Foreign Application Priority Data
Jul. 26, 2006 (DE) 10 2006 034 442.1

(51) Int. Cl. C07C 45/00; C07F 15/00; B01J 31/12

(52) U.S. Cl. 568/454; 556/13; 502/155

(57) ABSTRACT

The present invention relates to the preparation and use of stable catalyst precursors of rhodium complex catalysts.

The present invention relates to the preparation and use of catalyst precursors, in particular catalyst precursors of rhodium complex catalysts.

The use of homogeneous rhodium complex catalysts in various processes of industrial organic chemistry has been known for a long time. In particular, the use of rhodium-phosphine, -phosphite, -phosphonite or -phosphinite ligand complexes in processes for the hydroformylation of olefins has been adequately described. Many processes using catalysts comprising ligands having at least one phosphorus-oxygen bond have become known but have not yet experienced widespread use. This is presumably due to the fact that these catalysts are quite expensive and have only a low stability towards relatively high temperatures, oxygen and/or water.

In industrial processes, the active catalyst is frequently not introduced into the process in pure form for cost reasons and/or because of handling difficulties, but is instead prepared in the hydroformylation reactor from one or more suitable precursor(s) under the reaction conditions of the hydroformylation.

The suitability of a potential catalyst precursor depends on various factors. These factors include: commercial availability and price, storage stability, suitable handlability in respect of transport and introduction into the reactor, compatibility with cocatalysts, solubility in the desired reaction medium, rapid catalyst activation or rapid start of the reaction with a minimal induction period and the absence of adverse effects of the by-products formed in catalyst formation on the production plant or the yield of the reaction.

Precursors which already contain ligands which, because of a high complex formation constant, can be removed only with difficulty, e.g. triphenylphosphine from which the relatively stable HRh(TPP)₃(CO) is formed, are unfavourable for the activity and/or regioselectivity of the catalyst.

It was therefore an object of the present invention to provide an alternative catalyst precursor which preferably can be handled readily in respect of transport and introduction into the reactor and, in particular, has a good solubility in the desired reaction medium. In addition, the catalyst precursor should preferably be able to be converted easily into the active catalyst and have a good storage stability. In particular, no by-products which have an adverse effect on the production plant or on the catalyst stability and/or the reactivity and/or the selectivity should be formed when using the catalyst precursor, i.e. in catalyst formation.

It has surprisingly been found that the precursors of rhodium complex catalysts are very stable and are thus easy to handle when they have the structure I. These compounds are very suitable catalyst precursors since they have a very good solubility and the ligands in the compounds of the formula I can easily be displaced by ligands of the desired catalyst system.

The present invention accordingly provides a catalyst precursor comprising a rhodium complex of the formula I

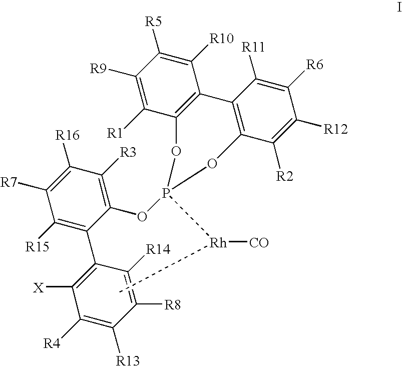

where R1 to R16 are identical or different and are each H, C₁-C₄-alkyl or C₁-C₄—O-alkyl radical or part of a ring system and X═O, S, NH or NR where R═C₁-C₁₀ alkyl radical, or a rhodium complex of the formula I and its use as catalyst precursor.

The present invention likewise provides a mixture containing a catalyst precursor according to the invention and also a process for preparing a catalyst precursor according to the invention, which is characterized in that a rhodium compound, for example RhH(CO)₂, dicarbonylrhodium acetylacetonate, and a CO source are added to a compound of the formula II

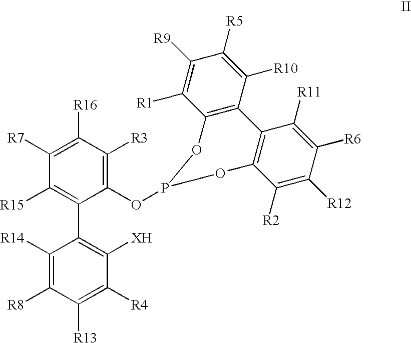

where R1 to R16 are identical or different and are each H, a C₁-C₄-alkyl or C₁-C₄—O-alkyl radical or part of a ring system and X═O, S, NH or NR where R═C₁-C₁₀-alkyl radical.

The present invention additionally provides for the use of a catalyst precursor according to the invention for preparing a catalyst for hydrocyanation, hydroacylation, hydroamidation, hydroesterification, aminolysis, alcoholysis, carbonylation, isomerization or a hydrogen transfer process and also a process for the hydroformylation of olefins, which is characterized in that a catalyst obtained from a catalyst precursor according to the invention is used.

If reference is made in the following to the catalyst precursor of the formula I, this always also encompasses the Rh complex of the formula I and its use as catalyst precursor.

The novel catalyst precursor of the formula I has the advantage that it has a high storage stability. In particular, the catalyst precursor has a relatively high stability towards thermal stress, oxidation or hydrolysis. As a result of the good storage stability, the catalyst precursor of the invention is highly suitable for being kept available as catalyst precursor for processes in which metal-organophosphorus ligands are to be used or have to be used. The appropriate metal-organophosphorus ligand complex catalysts can be produced very simply from the catalyst precursors of the invention by addition of the desired ligands.

The invention is described by way of example below, without the invention, whose scope is defined by the claims and the description, being restricted thereto. The claims themselves are also part of the disclosure content of the present invention. If ranges, general formulae or classes of compounds are indicated in the following, these encompass not only the corresponding ranges or groups of compounds which are explicitly mentioned but also all subranges and subgroups of compounds which can be obtained by leaving out individual values (ranges) or compounds.

The catalyst precursor of the invention is characterized by it comprising a rhodium complex of the formula I

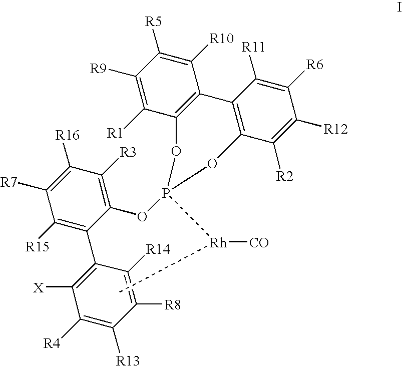

where R1 to R16 are identical or different and are each H, a C₁-C₄-alkyl or C₁-C₄—O-alkyl radical or part of a ring system and X═O, S, NH or NR where R═C₁-C₁₀-alkyl radical. The bond between X and the carbon atom to which X is bound can be a single or multiple bond. The radicals R1 to R16 can be identical or different, a plurality of the radicals R1 to R16 also being able to have the same meaning. The ring system can be formed by one or more of the radicals R1 to R16. The ring system can be aliphatic or heteroaliphatic or aromatic or heteroaromatic. The ring system is preferably fused onto the benzene ring. The ring system is preferably a fused-on aromatic ring system and is in each case formed by two adjacent radicals. For example, the radicals R5 and R10 and/or the radicals R11 and R6 can be joined and form a fused-on aromatic ring system. If, for example, both the radicals R5 and R10 and also the radicals R11 and R6 form such a system, the compound of the formula I can be a compound comprising a binaphthyl group.

The catalyst precursor of the invention preferably comprises a rhodium complex of the formula Ia

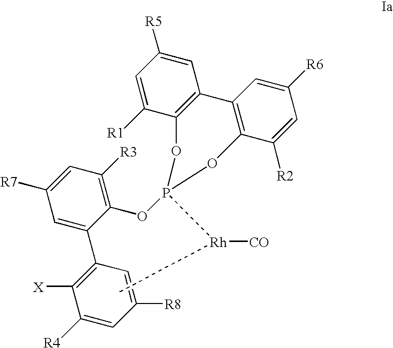

where R1 to R8 are identical or different and are each H, a C₁-C₄-alkyl or C₁-C₄—O-alkyl radical and X═O, S, NH or NR where R═C₁-C₁₀-alkyl radical.

Preference is given to at least one of the radicals R1 to R4 being a C₁-C₄-alkyl radical. Further preference is given to all the radicals R1 to R4 being a C₁-C₄-alkyl radical. It can be advantageous for at least one of the radicals R1 to R4 to be a tert-butyl radical. Very particular preference is given to all radicals R1 to R4 being a tert-butyl radical.

It can be advantageous for at least one of the radicals R5 to R8 to be a C₁-C₄—O-alkyl radical. Preference is given to all radicals R5 to R8 being a C₁-C₄—O-alkyl radical. Further preference is given to at least one of the radicals R5 to R8 being a methoxy radical. Particular preference is given to all radicals R5 to R8 being a methoxy radical.

The catalyst precursor of the invention is very particularly preferably a complex of the formula Ib.

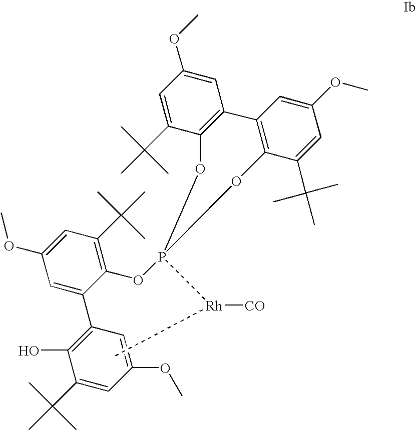

The molar ratio of rhodium to organophosphorus ligand can be from 1.1 to 0.9. The molar ratio of rhodium to organophosphorus ligand in the catalyst precursor of the invention is preferably 1:1.

The molar ratio of rhodium to CO can be from 1.1 to 0.9. The molar ratio of rhodium to CO in the catalyst precursor of the invention is preferably 1:1.

The catalyst precursor of the invention can be prepared, for example, by the process of the invention for preparing the catalyst precursor of the invention. The process of the invention for preparing a catalyst precursor according to the invention is characterized by a rhodium compound and a CO source being added to a compound of the formula II

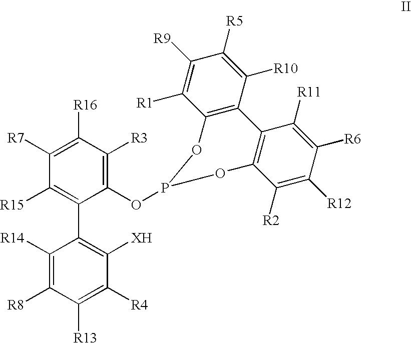

where R1 to R16 are identical or different and are each H, a C₁-C₄-alkyl or C₁-C₄—O-alkyl radical or part of a ring system and X═O, S, NH or NR where R═C₁-C₁₀-alkyl radical. The radicals R1 to R16 can be identical or different, with it also being possible for a plurality of the radicals R1 to R16 to have the same meanings. The ring system can be formed by one or more of the radicals R1 to R16. The ring system can be aliphatic or heteroaliphatic or aromatic or heteroaromatic. The ring system is preferably fused onto the benzene ring. The ring system is preferably a fused-on aromatic ring system and is formed by two adjacent radicals. For example, the radicals R5 and R10 and/or the radicals R11 and R6 can be joined and form a fused-on aromatic ring system. If, for example, both the radicals R5 and R10 and also the radicals R11 and R6 form such a system, the compound of the formula I can be, for example, a compound comprising a binaphthyl group.

Preference is given to using a compound of the formula IIa

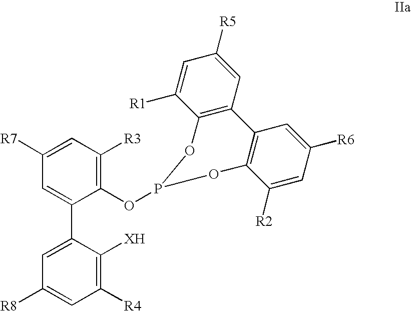

where R1 to R8=H, a C₁-C₄-alkyl or C₁-C₄—O-alkyl radical and X═O, S, NH or NR where R═C₁-C₁₀-alkyl radical as compound of the formula II. Particular preference is given to using a compound of the formula Ia in which the radicals R1, R2, R3 and R4 are tert-butyl radicals and the radicals R5, R6, R7 and R8 are methoxy radicals as compound of the formula II.

The CO source can be, for example, carbon monoxide gas itself. As rhodium compounds, it is possible to use, for example, rhodium nitrate, rhodium chloride, rhodium acetate, rhodium octanoate or rhodium nonanoate.

However, a rhodium carbonyl compound which simultaneously serves as CO source is preferably used as rhodium compound in the process of the invention. Such a rhodium compound can be, for example, dicarbonylrhodium acetylacetonate.

Compounds of the formula II can be prepared as described in the prior art. In particular, the preparation of compounds of the formula II can be carried out as described in PCT/EP2004/052729 and the references cited therein. The preparation of a compound of the formula II is described by way of example in Examples 1 to 3.

The catalyst precursor of the invention can be used as a pure substance or as a mixture. The mixtures according to the invention which contain the catalyst precursor of the invention can comprise, in particular, one or more solvents in addition to the catalyst precursor. Such solvents can be solvents which are inert in respect of the reaction for which the catalyst precursor is to be used after conversion into the catalyst. If one of the starting materials is used as solvent in the reactions, it can be advantageous to provide one of these starting materials used as solvent in the reaction as solvent in the mixture according to the invention. If the catalyst precursor is to be used, for example, for forming the catalyst for a hydroformylation reaction, it can be advantageous to use the olefin used in the hydroformylation, e.g. a C₄-, C₅-, C₆-, C₇-, C₈-, C₉-, C₁₀-, C₁₁-, C₁₂-, C₁₃-, C₁₄-, C₁₅-, C₁₆- or C₂₀-olefin, or the alcohol or aldehyde produced in the hydroformylation, e.g. C₅-, C₆-, C₇-, C₈-, C₉-, C₁₀-, C₁₁-, C₁₂, C₁₃, C₁₄-, C₅-, C₁₆-, C₁₇- or C₂₁-alcohol or -aldehyde, as solvent. If the mixture of the invention is to comprise an inert solvent, it is in the case of a hydroformylation possible to use, for example, toluene, diphyl, a commercially available mixture of biphenyl and diphenyl ether in a ratio of about 1:3), texanol, dioctyl phthalate, diisononyl phthalate, high boilers formed in the hydroformylation or propylene carbonate or butylene carbonate.

Apart from the catalyst precursors and, if desired, a solvent, the mixtures of the invention can contain further ligands, in particular organophosphorus ligands, or metal complexes comprising organophosphorus ligands.

The catalyst precursor of the invention can be used as precursor for preparing a catalyst for hydrocyanation, hydroacylation, hydroamidation, hydroesterification, aminolysis, alcoholysis, carbonylation, isomerization or a hydrogen transfer process. To produce the actual catalyst, it has been found to be advantageous to react the catalyst precursor with the ligand intended for the metal complex catalyst under reaction conditions, resulting in complete or at least partial ligand exchange taking place.

A process for the hydroformylation of olefins using a catalyst which has been obtained from a catalyst precursor according to the invention is described by way of example below.

In the process of the invention for the hydroformylation of olefins, preference is given to using olefins having from 2 to 25 carbon atoms, particularly preferably from 6 to 12 and very particularly preferably 8, 9, 10, 11 or 12 carbon atoms.

The complex catalysts which are prepared from the catalyst precursor and are used in the hydroformylation process can be compounds and complexes known from the prior art. They can be obtained by reacting the precursor of the invention with the desired ligand. Apart from the complex catalysts, free organophosphorus ligands can, if desired, be present in the reaction mixture of the hydroformylation. The complex catalysts or the free ligands preferably have/are ligands selected from among phosphines, phosphites, phosphinites, phosphonites. The ligands can have one or more phosphine, phosphite, phosphonite or phosphinite groups. It is likewise possible for the ligands to have two or more different groups selected from among phosphine, phosphite, phosphonite or phosphinite groups. In particular, the ligands can be bisphosphites, bisphosphines, bisphosphonites, bisphosphinites, phosphine-phosphites, phosphine-phosphonites, phosphine-phosphinites, phosphite-phosphonites, phosphite-phosphinites or phosphonite-phosphinites. The ligands of the complex catalyst and the free ligands can be identical or different. The organophosphorus ligands of the complex catalysts and the free ligands are preferably identical. Examples of complex catalysts or ligands which can be used and their preparation and use in hydroformylation may be found in, for example, EP 0 213 639, EP 0 214 622, EP 0 155 508, EP 0 781 166, EP 1209164, EP 1201675, DE 10114868, DE 10140083, DE 10140086, DE 10210918, which are expressly incorporated by reference.

### Examples of Preferred Ligands are:

Phosphines: triphenylphosphine, tris(p-tolyl)phosphine, tris(m-tolyl)phosphine, tris(o-tolyl)phosphine, tris(p-methoxyphenyl)phosphine, tris(p-dimethylamino-phenyl)phosphine, tris(cyclohexyl)phosphine, tris(cyclopentyl)phosphine, triethylphosphine, tris(1-naphthyl)phosphine, tribenzylphosphine, tri-n-butylphosphine, tri-t-butylphosphine.

Phosphites: trimethyl phosphite, triethyl phosphite, tri-n-propyl phosphite, tri-1-propyl phosphite, tri-n-butyl phosphite, tri-1-butyl phosphite, tri-t-butyl phosphite, tris(2-ethylhexyl) phosphite, triphenyl phosphite, tris(2,4-di-t-butylphenyl) phosphite, tris(2-t-butyl-4-methoxy-phenyl) phosphite, tris(2-t-butyl-4-methylphenyl) phosphite, tris(p-cresyl) phosphite.

Phosphonites: methyldiethoxyphosphine, phenyldimethoxyphosphine, phenyldiphenoxy-phosphine, 2-phenoxy-2H-dibenz[c,e][1,2]oxaphosphorin and derivatives thereof in which all or some of the hydrogen atoms have been replaced by alkyl and/or aryl radicals or halogen atoms.

Customary phosphinite ligands are diphenyl(phenoxy)phosphine and derivatives thereof, diphenyl(methoxy)phosphine and diphenyl(ethoxy)phosphine.

In the hydroformylation process of the invention, particular preference is given to using complex catalysts which comprise an organophosphorus ligand containing acyl phosphite or heteroacyl phosphite groups. Acyl phosphites or ligands having acyl phosphite groups, their preparation and their use in hydroformylation are described, for example, in DE 100 53 272, which is incorporated by reference into the disclosure of the present invention. Heteroacyl phosphites and ligands having heteroacyl phosphite groups, their preparation and their use in hydroformylation are described, for example, in DE 10 2004 013 514.

Among the acyl phosphites described in DE 100 53 272, the acyl phosphites shown below are particularly preferred organophosphorus ligands which can be present as catalyst complex ligand and/or as freed ligand in a hydroformylation process according to the invention.

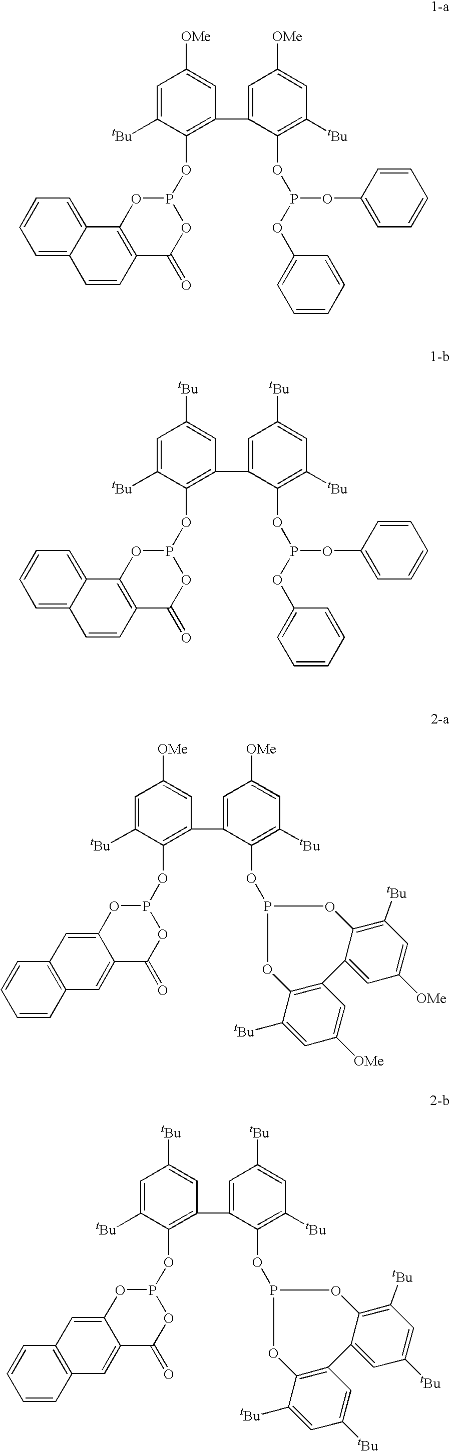

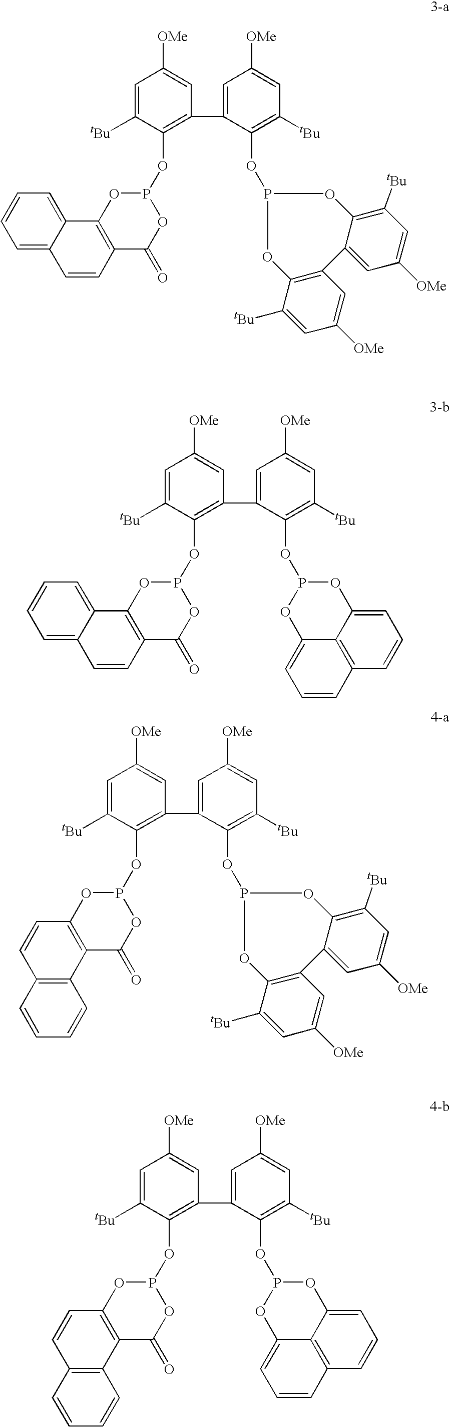

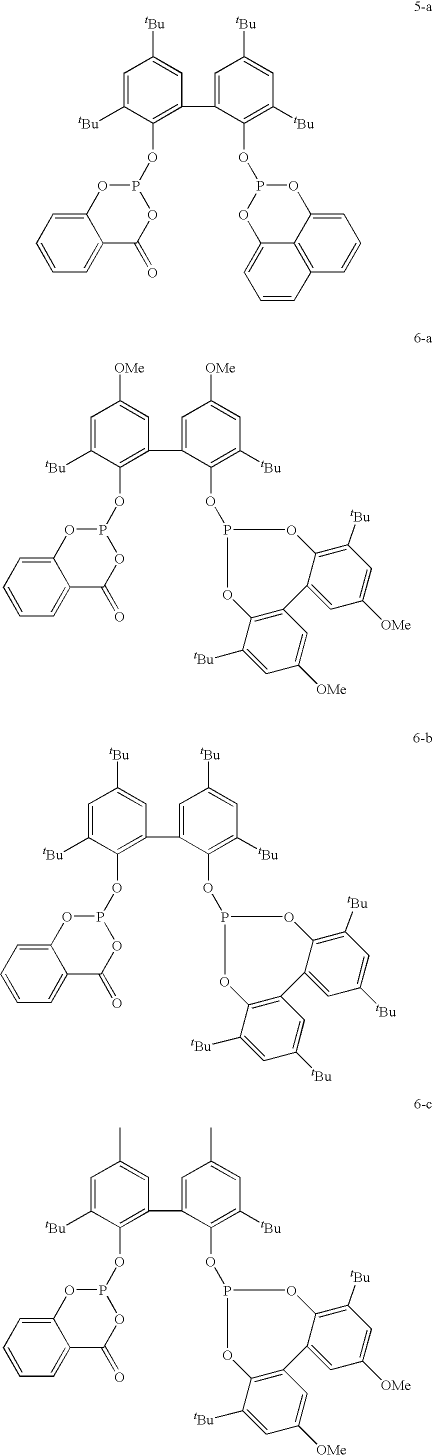

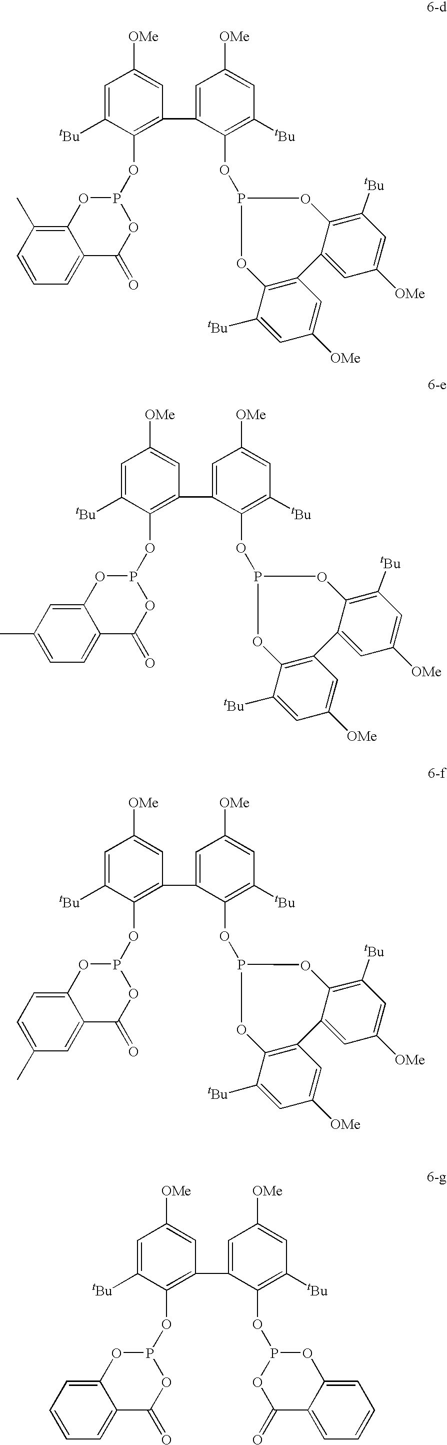

In a further preferred embodiment of the process of the invention, the heteroacyl phosphites of the general formula (I) described in DE 10 2004 013 514 can be used as ligands.

The hydroformylation process of the invention is preferably carried out using from 1 to 500 mol, preferably from 1 to 200 mol and particularly preferably from 2 to 50 mol, of organophosphorus ligand per mol of rhodium. Fresh organophosphorus ligands can be added at any point in time during the hydroformylation reaction in order to keep the concentration of free heteroacyl phosphite, i.e. heteroacyl phosphite which is not coordinated to the metal, constant.

The concentration of the metal in the hydroformylation mixture is preferably in the range from 1 ppm by mass to 1000 ppm by mass, more preferably in the range from 5 ppm by mass to 300 ppm by mass, based on the total mass of the hydroformylation mixture.

The hydroformylation reactions carried out using the organophosphorus ligands or the corresponding metal complexes can be carried out according to known methods as described, for example, in J. FALBE, “New Syntheses with Carbon Monoxide”, Springer Verlag, Berlin, Heidelberg, New York, page 95 ff., (1980). The olefin compound(s) is (are) reacted with a mixture of CO and H₂ (synthesis gas) in the presence of the catalyst to form the aldehyde having one more carbon atom.

The reaction temperatures are preferably from 40° C. to 180° C. and preferably from 75° C. to 140° C. The pressures under which the hydroformylation occurs are preferably from 0.1 to 30 MPa of synthesis gas and more preferably from 1 to 6.4 MPa. The molar ratio of hydrogen to carbon monoxide (H₂/CO) in the synthesis gas is preferably from 10/1 to 1/10 and more preferably from 1/1 to 2/1.

The catalyst or the ligand is preferably present as a homogeneous solution in the hydroformylation mixture comprising starting materials (olefins and synthesis gas) and products (aldehydes, alcohols, high boilers formed in the process). In addition, it is possible for a solvent to be present, the solvent also being able to be selected from among the starting materials (olefins) or products (aldehydes) of the reaction. Further possible solvents are organic compounds which do not interfere in the hydroformylation reaction and can preferably be separated off again easily, e.g. by distillation or extraction. Such solvents can be, for example, hydrocarbons such as toluene.

The starting materials for the hydroformylation are olefins or mixtures of olefins having from 2 to 25 carbon atoms and a terminal or internal C═C double bond. Preferred starting materials are, in particular, α-olefins such as propene, 1-butene, 2-butene, 1-hexene, 1-octene and also dimers and trimers of butene (isomer mixtures), in particular dibutene and tributene.

The hydroformylation can be carried out continuously or batchwise. Examples of industrial hydroformylation apparatuses are stirred vessels, bubble columns, jet nozzle reactors, tube reactors or loop reactors, some of which may be cascaded and/or provided with internals. The reaction can be carried out in a single stage or in a plurality of stages.

The work-up of the hydroformylation mixture can be carried out in various ways known from the prior art. The work-up is preferably carried out by firstly separating off all gaseous constituents from the hydroformylation mixture. The hydroformylation products and any unreacted starting olefins are then usually separated off. This separation can, for example, be achieved by the use of flash evaporators, falling film evaporators or distillation columns. A fraction which comprises essentially the catalyst and possibly high boilers formed as by-products can be obtained as residue. This fraction can be recirculated to the hydroformylation.

The following examples illustrate the invention without restricting its scope which is defined by the claims and the description.

### EXAMPLE 1

### Preparation of 3,3′-tert-butyl-2,2′-dihydroxy-5,5′-dimethoxybiphenyl Chlorophosphite (Compound III)

In a 500 ml Schlenk vessel, 35.8 g (0.1 mol) of 3,3′-tert-butyl-2,2′-dihydroxy-5,5′-dimethoxybiphenol (L001) and 30.7 g (42.3 ml; 0.3 mol) of dried and degassed triethylamine were dissolved in 350 ml of dried toluene. In a second Schlenk vessel (1000 ml), 13.9 g (8.8 ml; 0.1 mol) of phosphorus trichloride were dissolved in 350 ml of dried toluene and the diphenol/triethylamine/toluene solution prepared beforehand was slowly and steadily added dropwise to this solution at a temperature of from −5 to 0° C. while stirring vigorously. The abovementioned reactions were carried out under argon. The solution was allowed to warm to RT overnight. The solid formed was then allowed to settle. A sample of the supernatant solution was taken for GC/MS (check for complete conversion of chlorophosphite). The solid was then filtered off on a frit. The solution was used for the further synthesis (Example 2).

### EXAMPLE 2

### Reaction of Chlorophosphite with 3,3′-tert-butyl-2,2′-dihydroxy-5,5′-dimethoxybiphenyl (Compound IV) to Form Compound IIb

The reactions described below were carried out under argon. 35.8 g (0.1 mol) of the bisphenyl compound IV were placed in a 1 l Schlenk vessel, the vessel was evacuated again and argon was admitted. 300 ml of dried toluene and 14 ml (10.2 g; 0.1 mol) of triethylamine were subsequently added (by means of an argon-flushed syringe) while stirring. The Schlenk vessel was subsequently heated briefly to 50-60° C. while stirring vigorously. Although small amounts of bisphenyl compound have not gone into solution, the contents of the Schlenk vessel were used further.

The chlorophosphite solution (0.1 mol) of the compound III from Example 1 was then added dropwise over a period of 1.5 hours to the diol/toluene/triethylamine solution prepared above while stirring vigorously at RT. The mixture was subsequently allowed to react further for 30 minutes. The solid formed was allowed to settle before a check for complete conversion was made. A GC/MS analysis was carried out on the supernatant solution. The sample examined still contained significant amounts of chlorophosphite (starting material).

The Schlenk vessel was heated at 80° C. for 2 hours while stirring vigorously and another sample was subsequently taken for GC/MS analysis to check conversion. The sample examined no longer contained any chlorophosphite. The solid was subsequently filtered off on a frit. The solvent was removed under an oil pump vacuum and compound IIb was obtained as a solid.

### EXAMPLE 3

### Preparation of a Catalyst Precursor Ib According to the Invention

20 ml of 50 ml of cyclohexane were distilled off to remove traces of water. 2 g of [(CO)₂Rh(acac)] (acac=anion of acetylacetone) and 11.58 g of the compound IIb were added to the remaining 30 ml of cyclohexane. The molar ratio was 1:2. The mixture was refluxed for 5 hours under a blanket of nitrogen using an oil bath. After cooling to 60° C., the mixture was filtered. The filtrate was evaporated to dryness. For this purpose, the temperature was increased to 90° C. and the pressure was reduced to 20 hPa. After constant weight had been achieved, a mixture of compound Ib and compound IIb in a molar ratio of 1:1 was obtained.

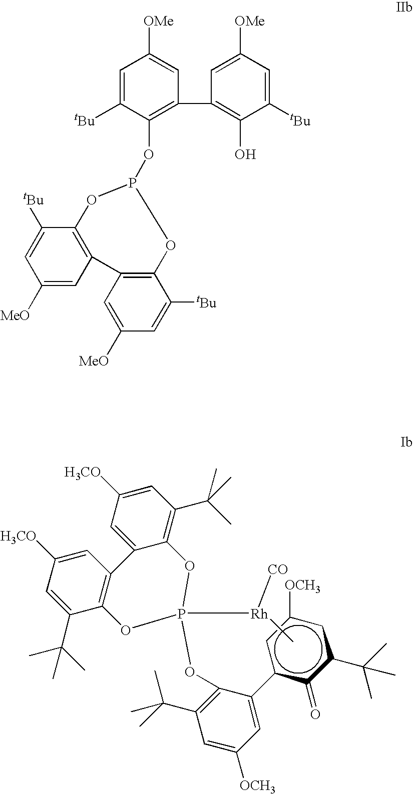

The pure complex can be isolated from this mixture by recrystallization. However, since compound IIb is inert in hydroformylation using the catalyst systems tested, mixtures prepared as described in Examples 1 to 3 were used in Example 4.

### EXAMPLE 4

### Solubilities

A number of experiments were carried out to test the solubility of various compounds used as catalyst precursors in various solvents. For this purpose, the solvents (10 ml) were placed in a jar having a snap-on lid. The Rh compounds were then added while stirring until the solution was saturated and the solid has clearly been seen. The saturated solution was subsequently placed on a hot plate and warmed slightly (without temperature monitoring to about 60° C.). The solutions were then allowed to stand overnight in the closed snap-on-lid jar. On the next day, the solutions above the sediment were then taken off at room temperature by means of a syringe filter. The solubilities were determined by analysis of the Rh concentration in the supernatant solution. The results of the solubility tests may be found in Tables 1 to 3.

TABLE 1
Solubility of catalyst precursors in isopropanol

| | Solubility in mg/kg |
|---|---|
| Catalyst precursor | (mg of Rh/kg of solvent) |
| Rh(acac)(CO)₂ | 2000 |
| HRh(triphenylphosphine)₃(CO) | 140 |
| Rh acetate | 500 |
| Rh nonanoate | 5100 |
| Ib | 6800 |

TABLE 2
Solubility of catalyst precursors in toluene

| | Solubility in mg/kg |
|---|---|
| Catalyst precursor | (mg of Rh/kg of solvent) |
| Rh(acac)(CO)₂ | 12 000 |
| HRh(triphenylphosphine)₃(CO) | 470 |
| Rh acetate | <10 |
| Rh nonanoate | 19 000 |
| Ib | 20 000 |

TABLE 3
Solubility of catalyst precursors in propylene carbonate

| | Solubility in mg/kg |
|---|---|
| Catalyst precursor | (mg of Rh/kg of solvent) |
| Rh(acac)(CO)₂ | 1100 |
| HRh(triphenylphosphine)₃(CO) | 530 |
| Rh acetate | 40 |
| Rh nonanoate | 30 |
| Ib | 4900 |

As can be seen from the results of the experiments in Example 4, a catalyst precursor according to the invention has a particularly good solubility in the selection of solvents tested (alcohol, aromatic and the highly polar propylene carbonate).

### EXAMPLE 5

### Hydroformylation of an Octene Mixture

The hydroformylation experiment was carried out in a 100 ml Parr autoclave provided with pressure regulator, gas flow measurement and propeller stirrer. The autoclave was charged under an argon atmosphere with 2.21×10⁻⁵ mol of rhodium in the form of [H Rh (6-a) CO] (preprepared catalyst with the ligand 6-a), 4.47×10⁻⁵ mol of the compound Iia (with X=oxygen) and about 29 g of toluene. About 29 g of n-octene mixture comprising about 1.5% by mass of 1-octene, 47% by mass of 2-octenes and 51.5% by mass of 3- and 4-octenes were placed in a pressure pipette. The molar ratio of Iia to Rh was thus about 20. The total mass of the reaction solution was thus about 58 g. After replacement of the argon atmosphere by flushing with synthesis gas (CO/H₂ 1:1), the reaction mixture was heated to 120° C. under synthesis gas pressure and while stirring (1000 rpm) and the precise intended pressure of 20 bar was then set. The synthesis gas pressure was kept constant by means of a pressure regulator during the entire duration of the reaction. After addition of the n-octene mixture, the gas consumption was recorded by means of a Hitec gas flow meter from Bronkhorst (NL). The reaction time of the hydroformylation experiment was 3 hours, with samples for GC analysis being taken from the autoclave at intervals. The reaction mixture was subsequently cooled to room temperature, the autoclave was vented and flushed with argon and a sample was then taken for GC analysis.

After 3 hours, a conversion of 60.0% and a selectivity to n-nonanal of 58.8% were obtained.

### EXAMPLE 6

### Comparative Experiment

The hydroformylation experiment was again carried out in a 100 ml Parr autoclave provided with pressure regulator, gas flow measurement and propeller stirrer. The autoclave was charged under an argon atmosphere with 2.14×10⁻⁵ mol of rhodium in the form of Rh nonanoate, 4.47×10⁻⁵ mol of ligand 6-a and about 29 g of toluene. About 29 g of n-octene mixture (composition as in Example 5) were placed in a pressure pipette. After replacement of the argon atmosphere by flushing with synthesis gas (CO/H₂ 1:1), the reaction mixture was heated to 120° C. under synthesis gas pressure and while stirring (1000 rpm) and the precise intended pressure of 20 bar was then set. The synthesis gas pressure was kept constant by means of a pressure regulator during the entire duration of the reaction. After addition of the olefin, the gas consumption was recorded by means of a Hitec gas flow meter from Bronkhorst (NL). The reaction time of the hydroformylation experiment was 3 hours. The reaction mixture was subsequently cooled to room temperature, the autoclave was vented and flushed with argon and a sample was then taken for GC analysis.

After 3 hours, a conversion of 59.6% and a selectivity to n-nonanal of 54.6% were obtained.

Examples 5 and 6 show that addition of the catalyst precursor IIa according to the invention does not have a negative influence on the result of the experiment.

1. A catalyst precursor comprising a rhodium complex of the formula I 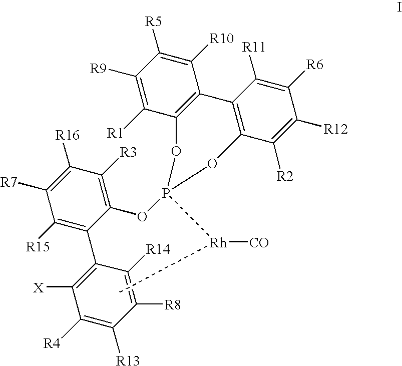
where R1 to R16 are identical or different and are each H, C₁-C₄-alkyl or C₁-C₄—O-alkyl radical or part of a ring system and X═O, S, NH or NR where R═C₁-C₁₀-alkyl radical.

2. A catalyst precursor according to claim 1,
characterized in that
the rhodium complex of the formula I is a rhodium complex of the formula Ia, 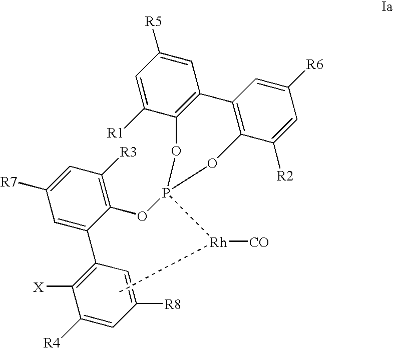
where R1 to R8 are identical or different and are each H, a C₁-C₄-alkyl or C₁-C₄—O-alkyl radical and X═O, S, NH or NR where R═C₁-C₁₀-alkyl radical.

3. A catalyst precursor according to claim 1,
characterized in that
at least one of the radicals R1 to R4 is a C₁-C₄-alkyl radical.

4. A catalyst precursor according to claim 3,
characterized in that
at least one of the radicals R1 to R4 is a tert-butyl radical.

5. A catalyst precursor according to claim 1,
characterized in that
at least one of the radicals R5 to R8 is a C₁-C₄—O-alkyl radical.

6. A catalyst precursor according to claim 5,
characterized in that
at least one of the radicals R5 to R8 is a methoxy radical.

7. A catalyst precursor according to claim 1,
characterized in that
the molar ratio of rhodium to organophosphorus ligand is from 1.1 to 0.9.

8. A mixture containing a catalyst precursor according to claim 1.

9. A process for preparing a catalyst precursor according to claim 1,
characterized in that
a rhodium compound and a CO source are added to a compound of the formula II 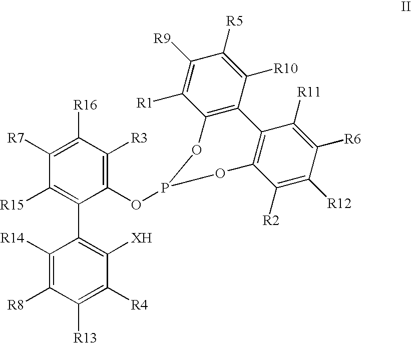
where R1 to R16 are identical or different and are each H, a C₁-C₄-alkyl or C₁-C₄—O-alkyl radical or part of a ring system and X═O, S, NH or NR where R═C₁-C₁₀-alkyl radical.

10. A process according to claim 9,
characterized in that
a compound of the formula IIa 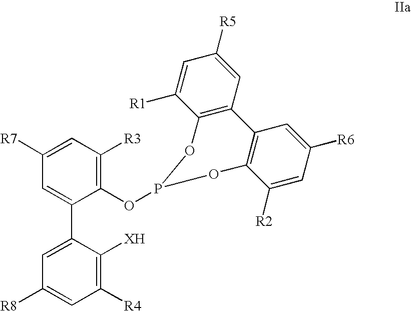
where R1 to R8 are identical or different and are each H, a C₁-C₄-alkyl or C₁-C₄—O-alkyl radical and X═O, S, NH or NR where R═C₁-C₁₀-alkyl radical, is used.

11. The use of a catalyst precursor according to claim 1 as precursor for preparing a catalyst for hydroformylation, hydrocyanation, hydroacylation, hydroamidation, hydroesterification, aminolysis, alcoholysis, carbonylation, isomerization or a hydrogen transfer process.

12. A process for the hydroformylation of olefins,
characterized in that
a catalyst obtained from a catalyst precursor according to claim 1 is used.
